(19) United States
(12) Patent Application Publication
(10) Pub. No.: US 2026/0060653 A1
(43) Pub. Date: Mar. 5, 2026

(54) MEDICAL DEVICE AND METHOD FOR MANUFACTURING MEDICAL DEVICE

(71) Applicant: ASAHI INTECC CO., LTD., Seto-shi (JP)

(72) Inventor: Yoshiki KATO, Seto-shi (JP)

(73) Assignee: ASAHI INTECC CO., LTD., Seto-shi (JP)

(21) Appl. No.: 19/383,815

(22) Filed: Nov. 10, 2025

Related U.S. Application Data
(63) Continuation of application No. PCT/JP2023/018778, filed on May 19, 2023.

(51) Int. Cl. A61B 8/00; A61B 8/12; A61M 25/00; A61M 25/01

(52) U.S. Cl. CPC A61B 8/445; A61B 8/12; A61M 25/0009; A61M 25/0026; A61M 25/0054; A61M 25/01; A61M 2025/0037; A61M 2025/0183

(57) ABSTRACT

A medical device includes a first tube, a second tube, a heat-shrinkable tube that bundles the first tube and the second tube, and a third tube in contact with an outer peripheral surface of the heat-shrinkable tube.

### CROSS REFERENCE TO RELATED APPLICATION

The present application claims priority to PCT/JP2023/018778 filed on May 19, 2023, the entire contents of which being incorporated herein by reference.

### TECHNICAL FIELD

The disclosed embodiments relate to a medical device and a method for manufacturing the medical device.

### BACKGROUND ART

There are known catheters used in percutaneous procedures. For example, Patent Literature 1 discloses a catheter that includes an observation portion lumen in which an observation portion used for observing the inside of a living body is provided, a first guide wire lumen provided closer to the distal end side than the observation portion, and a second guide wire lumen provided closer to the rear end side than the observation portion.

### CITATION LIST

### Patent Literature

Patent Literature 1: JP 2006-20944 A

### SUMMARY

### Technical Problems

Here, as the medical device, there is a device of a type configured by bundling a plurality of tubes in order to improve safety and operability while satisfying the required performance. Due to differences in the properties of the plurality of tubes, it is sometimes difficult to bundle the plurality of tubes at once. However, in the technique disclosed in Patent Literature 1, such a problem is not considered at all. Such a problem is not limited to a vascular system and is common to medical devices inserted into various organs in a human body, such as a lymphatic system, a biliary system, a urinary system, an airway system, a digestive system, a secretory gland, and a reproductive organ.

The disclosed embodiments have been made in order to solve at least a part of the above-described problem and has an object to provide a technique with which, in a medical device having a plurality of tubes, the plurality of tubes can be integrated even when there is a difference in the properties of the tubes.

### Solutions to Problems

The disclosed embodiments have been made to solve at least a part of the above-described and other issues and can be realized as the following aspects.
(1) According to an aspect of the disclosed embodiments, a medical device is provided. The medical device includes a first tube, a second tube, a heat-shrinkable tube that bundles the first tube and the second tube, and a third tube that is in contact with an outer peripheral surface of the heat-shrinkable tube.

It should be noted that the disclosed embodiments can be realized in various modes and, for example, can be realized in modes such as a medical device, a medical tube, a catheter, and manufacturing methods thereof.

### BRIEF DESCRIPTION OF DRAWINGS

FIG. 1 is an explanatory view illustrating a configuration of a medical device.

FIG. 2 is an explanatory view illustrating a configuration of the medical device.

FIGS. 3A to 3E are transverse sectional views of a catheter.

FIG. 4 is a diagram illustrating a method of using the catheter.

FIG. 5 is a diagram illustrating a method of using the catheter.

FIG. 6 is an enlarged view of a part of the catheter in the vicinity of a heat-shrinkable tube.

FIG. 7 is a transverse sectional view of the catheter taken along the line F-F in FIG. 6.

FIGS. 8A to 8D are diagrams illustrating a method for manufacturing the catheter.

FIGS. 9A and 9B are diagrams illustrating the method for manufacturing the catheter.

FIG. 10 is a transverse sectional view of a catheter according to a second embodiment.

FIG. 11 is a transverse sectional view of a catheter according to a third embodiment.

FIG. 12 is an enlarged view of a part of a catheter according to a fourth embodiment in the vicinity of the heat-shrinkable tube.

### DETAILED DESCRIPTION

### First Embodiment

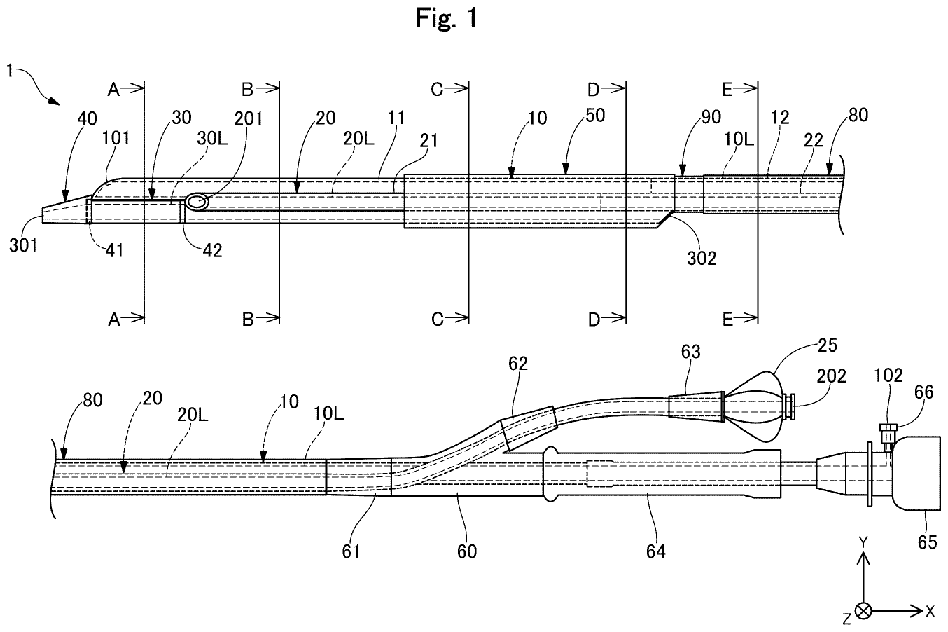

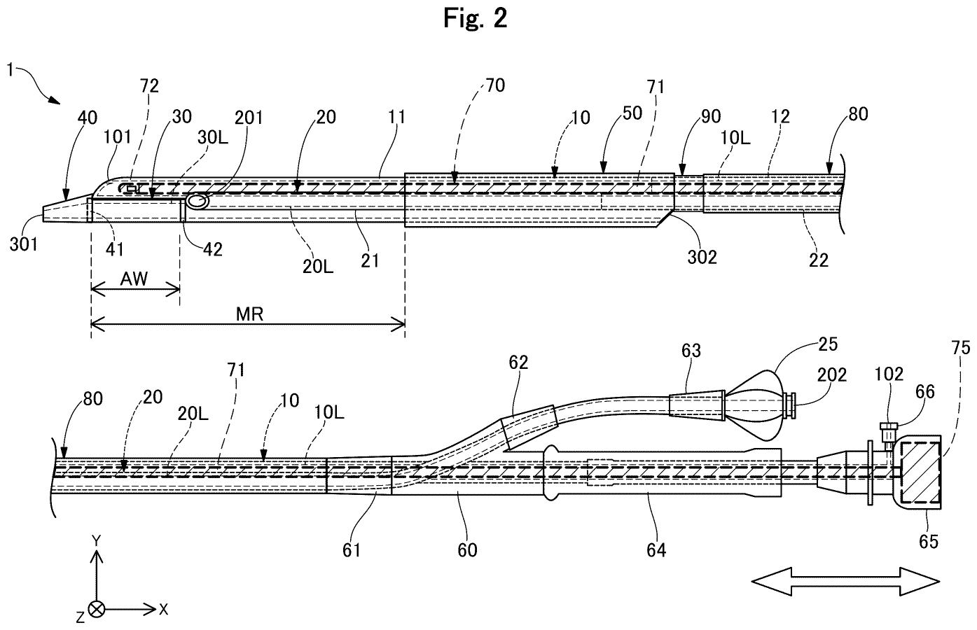

FIGS. 1 and 2 are explanatory views illustrating a configuration of a medical device 1. The medical device 1 according to the present embodiment is, for example, a catheter used to treat a lesion in a living body lumen such as a CTO occurring in a blood vessel. Hereinafter, the medical device 1 is also referred to as the “catheter 1”. As illustrated in FIGS. 1 and 2, the catheter 1 includes a sensor tube 10, an OTW (Over The Wire) tube 20, an RX (Rapid Exchange) tube 30, a distal tip 40, a first marker 41, a second marker 42, a first outer tube 50, a branch connector 60, a first reinforcing member 61 to a third reinforcing member 63, a cylindrical member 64, a connector 65, a connector 25, a sensor 70, a second outer tube 80, and a heat-shrinkable tube 90. The sensor tube 10 is also referred to as a “medical device” or a “medical tube”. The OTW tube 20 is also referred to as a “medical device” or a “medical tube”.

In FIG. 1, the sensor 70 is not illustrated in order to explain the configurations of the tube and the lumen in the tube. In FIG. 2, the sensor 70 incorporated in a sensor lumen 10L in the sensor tube 10 is indicated by a broken line and hatched with oblique lines.

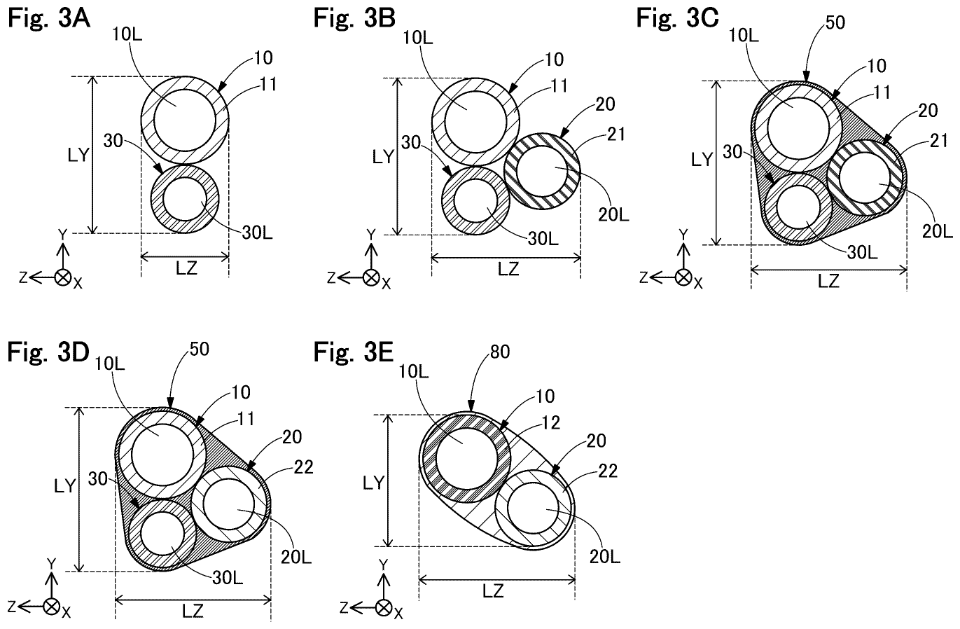

In FIGS. 1 and 2, for convenience of description, the relative ratio of the size of each constituent member includes a portion different from the actual portion. Further, a part of each constituent member includes an exaggerated portion. FIGS. 1 and 2 illustrate the XYZ axes orthogonal to each other. The X-axis corresponds to the longitudinal direction of the catheter 1, the Y-axis corresponds to the height direction of the catheter 1, and the Z-axis corresponds to the width direction of the catheter 1. The left side (−X-axis direction) of FIGS. 1 and 2 is referred to as the “distal end side” of the catheter 1 and each constituent member, and the right side (+X-axis direction) of FIGS. 1 and 2 is referred to as the “proximal end side” of the catheter 1 and each constituent member. In addition, among both ends of the catheter 1 and each constituent member in the longitudinal direction (X-axis direction), one end located on the distal end side is referred to as the “distal end”, and the other end located on the proximal end side is referred to as the “proximal end”. The distal end and the vicinity thereof are referred to as the “distal end portion”, and the proximal end and the vicinity thereof are referred to as the “proximal end portion”. The distal end side is inserted into a living body, and the proximal end side is operated by a professional such as a doctor. These points are common to FIGS. 3A to 3E and subsequent drawings.

FIGS. 3A to 3E are transverse sectional views of the catheter 1. FIG. 3A illustrates a transverse section of the catheter 1 taken along the line A-A in FIG. 1. FIG. 3B illustrates a transverse section of the catheter 1 taken along the line B-B in FIG. 1. FIG. 3C illustrates a transverse section of the catheter 1 taken along the line C-C in FIG. 1. FIG. 3D illustrates a transverse section of the catheter 1 taken along the line D-D in FIG. 1. FIG. 3E illustrates a transverse section of the catheter 1 taken along the line E-E in FIG. 1. Hereinafter, the configuration of the catheter 1 will be described with reference to FIGS. 1 to 3E.

The sensor tube 10 is a hollow cylindrical member (tubular body) having an elongated outer shape. The sensor tube 10 extends linearly along the longitudinal direction (X-axis direction) of the catheter 1 in parallel with the OTW tube 20 and the RX tube 30. The sensor lumen 10L (broken line) for accommodating the sensor 70 is formed inside the sensor tube 10. The sensor lumen 10L is a lumen for the sensor 70.

In the longitudinal direction of the catheter 1, the distal end of the sensor tube 10 is located at the same position as the distal end of the RX tube 30 or slightly closer to the proximal end side than the distal end of the RX tube 30. A distal end opening 101 communicating between the distal end of the sensor lumen 10L and the outside is formed at the distal end of the sensor tube 10. The distal end opening 101 is a fluid discharge port for bringing the inside of the sensor lumen 10L into a wet state. The proximal end of the sensor tube 10 is located closer to the proximal end side than the proximal end of the OTW lumen 20L and the proximal end of the RX tube 30 in the longitudinal direction of the catheter 1. The first reinforcing member 61, the branch connector 60, the cylindrical member 64, and the connector 65 are attached on the proximal end side of the sensor tube 10 from the distal end side toward the proximal end side. Details will be described below. A fluid supply portion 66 is attached to the connector 65, and a proximal end opening 102 communicating between the proximal end of the sensor lumen 10L and the outside is formed in the fluid supply portion 66. The proximal end opening 102 is a fluid supply port to the sensor lumen 10L.

As illustrated in FIG. 1, the sensor tube 10 includes a distal end side tube 11 provided on the distal end side and a proximal end side tube 12 provided closer to the proximal end side than the distal end side tube 11. Both the distal end side tube 11 and the proximal end side tube 12 are hollow cylindrical members (tubular bodies) having an elongated outer shape. The distal end side tube 11 and the proximal end side tube 12 are connected to each other inside the first outer tube 50 in the longitudinal direction. Specifically, both the distal end side tube 11 and the proximal end side tube 12 form a part of the sensor lumen 10L.

The OTW tube 20 is a hollow cylindrical member (tubular body) having an elongated outer shape. On the distal end side of the branch connector 60, the OTW tube 20 extends linearly along the longitudinal direction of the catheter 1 in parallel with the sensor tube 10 and the RX tube 30. An OTW lumen 20L (broken line) for accommodating a therapeutic device (for example, a plasma guide wire or a penetration guide wire) is formed inside the OTW tube 20. The OTW lumen 20L has no proximal end opening in a portion that is located in the living body lumen when the catheter 1 is used. The OTW lumen 20L is an over-the-wire (OTW) type lumen.

The distal end of the OTW tube 20 is located closer to the proximal end side than the distal end of the sensor tube 10 and the distal end of the RX tube 30 in the longitudinal direction of the catheter 1. A distal end opening 201 communicating between the distal end of the OTW lumen 20L and the outside is formed at the distal end of the OTW tube 20. The distal end opening 201 is a device projection port for projecting a therapeutic device toward a living tissue. Since the distal end portion of the OTW tube 20 is obliquely cut, the distal end opening 201 is oriented in a direction intersecting with the longitudinal direction of the catheter 1. Thus, when the catheter 1 is used, the therapeutic device can easily reach the living tissue present around the catheter 1. The proximal end of the OTW tube 20 is located closer to the distal end side than the proximal end of the sensor tube 10 and closer to the proximal end side than the proximal end of the RX tube 30 in the longitudinal direction of the catheter 1. The first reinforcing member 61, the branch connector 60, the second reinforcing member 62, the third reinforcing member 63, and the connector 25 are attached on the proximal end side of the OTW tube 20 from the distal end side toward the proximal end side. Details will be described below. A proximal end opening 202 communicating between the proximal end of the OTW lumen 20L and the outside is formed in the connector 25. The proximal end opening 202 is a device insertion port for inserting a therapeutic device into the OTW lumen 20L.

As illustrated in FIG. 1, the OTW tube 20 includes a distal end side tube 21 provided on the distal end side and a proximal end side tube 22 provided closer to the proximal end side than the distal end side tube 21. Both the distal end side tube 21 and the proximal end side tube 22 are hollow cylindrical members (tubular bodies) having an elongated outer shape. The distal end side tube 21 and the proximal end side tube 22 are connected to each other inside the first outer tube 50 in the longitudinal direction. That is, both the distal end side tube 21 and the proximal end side tube 22 form a part of the OTW lumen 20L.

The RX tube 30 is a hollow cylindrical member (tubular body) having an elongated outer shape. The RX tube 30 extends linearly along the longitudinal direction of the catheter 1 in parallel with the sensor tube 10 and the OTW tube 20. An RX lumen 30L (broken line) for accommodating a work hose wire is formed inside the RX tube 30. The work hose wire is a guide wire that is inserted to the vicinity of a lesion in advance of the catheter 1 in order to deliver the catheter 1.

The distal end of the RX tube 30 is located at the same position as the distal end of the sensor tube 10 or slightly closer to the distal end side than the distal end of the sensor tube 10 in the longitudinal direction of the catheter 1. The hollow distal tip 40 is joined to the distal end portion of the RX tube 30. A distal end opening 301 communicating between the distal end of the RX lumen 30L and the outside is formed at the distal end of the distal tip 40. The distal end opening 301 is a wire insertion port for inserting a work hose wire into the RX lumen 30L. The proximal end of the RX tube 30 is located closer to the distal end side than the proximal end of the sensor tube 10 and the proximal end of the OTW tube 20 in the longitudinal direction of the catheter 1. A proximal end opening 302 communicating between the proximal end of the RX lumen 30L and the outside is formed at the proximal end of the RX tube 30. The proximal end opening 302 is a wire drawing port for drawing out the work hose wire to the outside. Since the proximal end of the RX tube 30 is obliquely cut, the proximal end opening 302 is oriented in a direction intersecting with the longitudinal direction of the catheter 1. Thus, when the catheter 1 is used, the work hose wire can be easily drawn out from the proximal end opening 302.

The distal tip 40 has radiopacity and is a cylindrical member in which the outer diameter expands from the distal end side toward the proximal end side. The distal tip 40 is joined to the distal end portion of the RX tube 30 and is thus located at the distal end of the catheter 1 and advances in the living body lumen prior to the other members. The inner cavity of the distal tip 40 communicates with the RX lumen 30L of the RX tube 30 and, as described above, the distal end opening 301 communicating between the distal end of the RX lumen 30L and the outside is formed at the distal end of the distal tip 40.

The first marker 41 and the second marker 42 are annular members having radiopacity. The first marker 41 is provided such that the proximal end of the first marker 41 and the proximal end of the distal tip 40 are at the same position in the longitudinal direction of the catheter 1. The first marker 41 is embedded between the outer peripheral surface of the RX tube 30 and the inner peripheral surface of the distal tip 40. The second marker 42 is provided such that the proximal end of the second marker 42 and the distal end of the distal end opening 201 are at the same position in the longitudinal direction of the catheter 1. The second marker 42 is joined to the outer peripheral surface of the RX tube 30. For joining the first marker 41 and the second marker 42, for example, joining between resins by thermal melting or joining with an adhesive such as an epoxy-based adhesive can be employed. The second marker 42 is visible to the naked eye. As described above, by arranging the first marker 41 and the second marker 42 on the RX tube 30, it is possible to prevent the first marker 41 and the second marker 42 from interfering with sensing (acquisition of image information) by the sensor 70.

As illustrated in FIG. 3A, in the transverse section taken along the line A-A, the sensor tube 10 (specifically, the distal end side tube 11) and the RX tube 30 are provided, and the outer peripheral surfaces thereof are joined to each other. As illustrated in FIG. 3B, in the transverse section taken along the line B-B, the sensor tube 10 (specifically, the distal end side tube 11), the OTW tube 20 (specifically, the distal end side tube 21), and the RX tube 30 are provided, and the outer peripheral surfaces thereof are joined to each other. As illustrated in FIG. 3C, in the transverse section taken along the line C-C, the sensor tube 10 (specifically, the distal end side tube 11), the OTW tube 20 (specifically, the distal end side tube 21), and the RX tube 30 are covered with the first outer tube 50. Specifically, the outer peripheral surfaces of the three tubes 10, 20, and 30 are covered with the first outer tube 50 formed by melting, and thus the three tubes 10, 20, and 30 are integrally fixed. As illustrated in FIG. 3D, in the transverse section taken along the line D-D, the sensor tube 10 (specifically, the distal end side tube 11), the OTW tube 20 (specifically, the proximal end side tube 22), and the RX tube 30 are covered with the first outer tube 50, as in FIG. 3C. As illustrated in FIG. 3E, in the transverse section taken along the line E-E, the sensor tube 10 (specifically, the proximal end side tube 12) and the OTW tube 20 (specifically, the proximal end side tube 22) are covered with the second outer tube 80. Specifically, the outer peripheral surfaces of the two tubes 10 and 20 are covered with the second outer tube 80 formed by melting, and thus the two tubes 10 and 20 are integrally fixed.

In the A-A transverse section and the B-B transverse section, the sensor tube 10, the OTW tube 20, and the RX tube 30 may be joined together by using any bonding material such as an epoxy-based adhesive, or may be welded by heat. In the A-A transverse section, the B-B transverse section, the C-C transverse section, and the D-D transverse section, a height LY of the catheter 1 is greater than a width LZ of the catheter 1. On the other hand, in the E-E transverse section, the height LY of the catheter 1 is smaller than the width LZ of the catheter 1. As illustrated in FIGS. 3A to 3E, the size relationship among the outer diameters of the three tubes 10, 20, and 30 is the outer diameter of the sensor tube 10>the outer diameter of the OTW tube 20>the outer diameter of the RX tube 30. In addition, the size relationship among the inner diameters (lumens) of the three tubes 10, 20, and 30 is the inner diameter of the sensor lumen 10L>the inner diameter of the OTW lumen 20L>the inner diameter of the RX lumen 30L. The size relationship among the outer diameters and the inner diameters is merely an example, and may be arbitrarily changed.

The A-A transverse section and the B-B transverse section, in other words, the outer shape of the catheter 1 closer to the distal end side than the first outer tube 50 is the shape along the contour of the two tubes 10 and 30 (or the three tubes 10, 20, and 30) arranged adjacent to each other, and a constricted portion (recess portion) is formed in the adjacent portion of each tube. The C-C transverse section and the D-D transverse section, in other words, the outer shape of the catheter 1 in the portion covered with the first outer tube 50 is a triangular shape with round corners (a round-corner triangular shape). The E-E transverse section, in other words, the outer shape of the catheter 1 in the portion covered with the second outer tube 80 is elliptical.

With reference back to FIG. 1, the description will be continued. The three tubes 10, 20, and 30 (specifically, the sensor tube 10, the OTW tube 20, and the RX tube 30) are fixed by the three tubes 90, 50, and 80.

The heat-shrinkable tube 90 is provided between the first outer tube 50 and the second outer tube 80 in the longitudinal direction of the catheter 1. The heat-shrinkable tube 90 covers the sensor tube 10 (specifically, a part of the proximal end side tube 12 on the distal end side) and the OTW tube 20 (specifically, a part of the proximal end side tube 22 on the distal end side) to bundle the two tubes 10 and 20. The heat-shrinkable tube 90 does not cover the RX tube 30, and the RX tube 30 is provided along the outer peripheral surface of the heat-shrinkable tube 90 in a state where the outer peripheral surface of the heat-shrinkable tube 90 and the outer peripheral surface of the RX tube 30 are in contact with each other. In other words, the heat-shrinkable tube 90 is disposed around and bundles the first tube 10 and the second tube 20, forming a sub-assembly. The third tube 30 is arranged adjacent to and in contact with an outer peripheral surface of the heat-shrinkable tube of the sub-assembly. The distal end of the heat-shrinkable tube 90 is located closer to the proximal end side than the distal end of the first outer tube 50 and closer to the distal end side than the proximal end opening 302. That is, the distal end portion of the heat-shrinkable tube 90 is covered with the first outer tube 50. The proximal end of the heat-shrinkable tube 90 is located closer to the proximal end side than the distal end of the second outer tube 80 and closer to the distal end side than the first reinforcing member 61. That is, the proximal end portion of the heat-shrinkable tube 90 is covered with the second outer tube 80. In other words, an intermediate portion of the heat-shrinkable tube 90 in the longitudinal direction of the catheter 1 is not covered with the first outer tube 50 or the second outer tube 80.

The first outer tube 50 is located closer to the distal end side than the heat-shrinkable tube 90 in the longitudinal direction of the catheter 1. The first outer tube 50 is provided in a section which is closer to the proximal end side than the distal end opening 201 and in which the three tubes 10, 20, and 30 extend side by side. In the example of FIG. 1, the distal end of the first outer tube 50 is located near the center between the distal end opening 201 and the proximal end opening 302. The proximal end of the first outer tube 50 is located near the proximal end of the proximal end opening 302. As described above, the first outer tube 50 is preferably provided at a position away from the distal end opening 201 toward the proximal end side. This can prevent the first outer tube 50 from interfering with sensing (acquisition of image information) by the sensor 70 inserted into the sensor lumen 10L. The first outer tube 50 covers and fixes the distal end portion of the heat-shrinkable tube 90, the sensor tube 10 (specifically, a part of the distal end side tube 11 on the proximal end side) exposed from the distal end of the heat-shrinkable tube 90, the OTW tube 20 (specifically, a part of the distal end side tube 21 on the proximal end side and a part of the proximal end side tube 22 on the distal end side) exposed from the distal end of the heat-shrinkable tube 90, and the RX tube 30. As illustrated in FIGS. 3C and 3D, the first outer tube 50 is substantially triangular, e.g., has a triangular outer shape with round corners, and has a thick portion formed by melting along the outer peripheral surfaces of the three tubes 10, 20, and 30.

The second outer tube 80 is located closer to the proximal end side than the heat-shrinkable tube 90 in the longitudinal direction of the catheter 1. The second outer tube 80 is provided in a section which is closer to the proximal end side than the proximal end opening 302 and in which the two tubes 10 and 20 extend side by side. In the example of FIG. 1, the distal end of the second outer tube 80 is located at a portion slightly away from the proximal end opening 302 toward the proximal end side. The proximal end of the second outer tube 80 is located inside the first reinforcing member 61. The second outer tube 80 covers and fixes the proximal end portion of the heat-shrinkable tube 90, the sensor tube 10 exposed from the proximal end of the heat-shrinkable tube 90 (specifically, a part of the proximal end side tube 12 on the proximal end side), and the OTW tube 20 exposed from the proximal end of the heat-shrinkable tube 90 (specifically, a part of the proximal end side tube 22). As illustrated in FIG. 3E, the second outer tube 80 has an elliptical outer shape and has a thick portion formed by melting along the outer peripheral surfaces of the two tubes 10 and 20.

The branch connector 60 is a member having a bifurcated inner cavity and is provided on the proximal end side of the catheter 1. The OTW tube 20 is inserted into one inner cavity of the branch connector 60. The sensor tube 10 is inserted into the other inner cavity of the branch connector 60. The first reinforcing member 61 is a hollow cylindrical member provided closer to the distal end side than the branch connector 60. The first reinforcing member 61 covers the outer periphery of the second outer tube 80 in which the sensor tube 10 and the OTW tube 20 are bundled, thereby reinforcing the distal end side of the branch connector 60.

The second reinforcing member 62 is a hollow cylindrical member provided closer to the proximal end side than one branch of the branch connector 60. The second reinforcing member 62 covers the outer periphery of the OTW tube 20 inserted into the branch connector 60, thereby reinforcing the proximal end side of the branch connector 60. The third reinforcing member 63 is a hollow cylindrical member provided closer to the distal end side than the connector 25. The third reinforcing member 63 covers the outer periphery of the OTW tube 20 inserted into the connector 25, thereby reinforcing the distal end side of the connector 25. The connector 25 is a member joined to the proximal end portion of the OTW tube 20. The connector 25 includes a pair of blade portions to be gripped by the professional. The proximal end opening 202 (device insertion port) communicating between the proximal end of the OTW lumen 20L and the outside is formed at the proximal end of the connector 25.

The cylindrical member 64 is a hollow cylindrical member provided closer to the proximal end side than the other branch of the branch connector 60. The cylindrical member 64 covers the outer periphery of the sensor tube 10 inserted into the branch connector 60, thereby reinforcing the proximal end side of the branch connector 60. The connector 65 is a member joined to the proximal end portion of the sensor tube 10. A housing for accommodating a connection terminal 75 of the sensor 70 is provided on the proximal end side of the connector 65. The fluid supply portion 66, in which the proximal end opening 102 communicating between the proximal end of the sensor lumen 10L and the outside is formed, is provided on the outer peripheral surface of the connector 65.

The sensor 70 (FIG. 2) is an imaging sensor that acquires image information. As illustrated in FIG. 2, the sensor 70 includes a main body part 71, a probe 72, and a connection terminal 75. The main body part 71 is an elongated member extending along the longitudinal direction of the catheter 1. A driving cable (coaxial line) that electrically connects the probe 72 and the connection terminal 75 is incorporated inside the main body part 71. The probe 72 includes an ultrasonic probe (also referred to as an ultrasonic vibrator, a piezoelectric body, an ultrasonic transmission/reception element, or an ultrasonic element) that transmits an ultrasonic wave toward a living tissue and receives the ultrasonic wave propagated through the living tissue and reflected. The probe 72 is also referred to as an imaging core or a transducer. The connection terminal 75 is a terminal that electrically connects the sensor 70 to a console terminal provided outside. The connection terminal 75 is provided at the proximal end of the main body part 71 and is accommodated in the housing of the connector 65.

The sensor 70 is electrically connected to an external console terminal via the connection terminal 75, receives the power supplied from the console terminal, and outputs a detection signal by the probe 72 to the console terminal. Thus, the console terminal can display the image information based on the detection signal of the probe 72. As illustrated in FIG. 2, the sensor 70 is fixed to the connector 65. Further, as indicated by the white arrow in FIG. 2, the professional grips the connector 65 and slides the connector 65 in the front-rear direction (the direction of the white arrow) and thus can move the position of the probe 72 of the sensor 70 within a range MR from the distal end of the sensor lumen 10L to the distal end of the first outer tube 50, in other words, within the predetermined range MR including the distal end opening 201. Hereinafter, the range MR is also referred to as the “movable range MR”. Further, a portion of the catheter 1 which is particularly suitable for sensing (acquisition of image information) by the sensor 70 is also referred to as an “acoustic window AW”. As illustrated in FIG. 2, the acoustic window AW is a section between the first marker 41 and the second marker 42 in the catheter 1.

The distal end side tube 11 of the sensor tube 10, the distal end side tube 21 of the OTW tube 20, and the RX tube 30 can be formed of a flexible material, for example, a thermoplastic resin such as a polyethylene resin, a polypropylene resin, or polyurethane, polyvinyl chloride, ethylene-vinyl acetate copolymers, cross-linked ethylene-vinyl acetate copolymers, polyamide elastomer, polyolefin elastomer, polyurethane elastomer, silicone rubber, or latex rubber. The distal end side tube 11 of the sensor tube 10, the distal end side tube 21 of the OTW tube 20, and the RX tube 30 may be formed of the same material or different materials.

The proximal end side tube 12 of the sensor tube 10 and the proximal end side tube 22 of the OTW tube 20 can be formed of, for example, a resin having high rigidity such as a nylon resin, a polyester resin, or a PEEK resin. The melting points of the proximal end side tube 12 of the sensor tube 10 and the proximal end side tube 22 of the OTW tube 20 are higher than the melting points of the above-described tubes 11, 21, and 30. The proximal end side tube 12 of the sensor tube 10 and the proximal end side tube 22 of the OTW tube 20 may be formed of the same material or different materials.

In the catheter 1 according to the present embodiment, by providing the section in which a part of the RX tube 30 having flexibility on the proximal end side is overlapped with the proximal end side tube 12 and the proximal end side tube 22 having a high rigidity (FIG. 1), gradual changes in the rigidity of the catheter 1 is achieved. The gradual changes in the rigidity of the catheter 1 may be rephrased as reducing a rigidity gap of the catheter 1. As a result, kink of the catheter 1 can be suppressed. Further, any one or more of the distal end side tube 11 and the proximal end side tube 12 of the sensor tube 10, the distal end side tube 21 and the proximal end side tube 22 of the OTW tube 20, and the RX tube 30 may have a multilayer structure in which tubes made of different materials are stacked.

The distal tip 40, the first marker 41, and the second marker 42 can be formed of a resin material or a metal material having radiopacity. For example, when a radiopaque resin material is used, it can be formed by mixing a radiopaque material such as bismuth trioxide, tungsten, or barium sulfate with a polyamide resin, a polyolefin resin, a polyester resin, a polyurethane resin, a silicone resin, or a fluororesin. For example, when a radiopaque metal material is used, it can be formed of gold, platinum, tungsten, or an alloy containing these elements (for example, a platinum-nickel alloy). The distal tip 40, the first marker 41, and the second marker 42 may be formed of the same material or different materials.

The branch connector 60, the first reinforcing member 61 to the third reinforcing member 63, the cylindrical member 64, the connector 65, and the connector 25 can be formed of a known resin material. The branch connector 60, the first reinforcing member 61 to the third reinforcing member 63, the cylindrical member 64, the connector 65, and the connector 25 may be formed of the same material or different materials.

The heat-shrinkable tube 90 is formed of a nylon-based elastomer resin (for example, polyamide elastomer) having thermoplasticity. The heat-shrinkable tube 90 has a property of shrinking without melting when heated in a prescribed temperature range. In addition, the heat-shrinkable tube 90 improves adhesiveness (property of easily sticking to another substance) at the time of heating as compared to that at the time of non-heating. The heat-shrinkable tube 90 may be formed of polyolefin, FEP (Fluorinated Ethylene Propylene), or silicone.

The first outer tube 50 and the second outer tube 80 are formed of a nylon-based elastomer resin having thermoplasticity. Unlike the heat-shrinkable tube 90, the first outer tube 50 and the second outer tube 80 have a property of melting when heated. In the example according to the present embodiment, a resin having lower shore hardness than the second outer tube 80 is used for the first outer tube 50. The first outer tube 50 and the second outer tube 80 may be formed of the same material or may be formed of different materials.

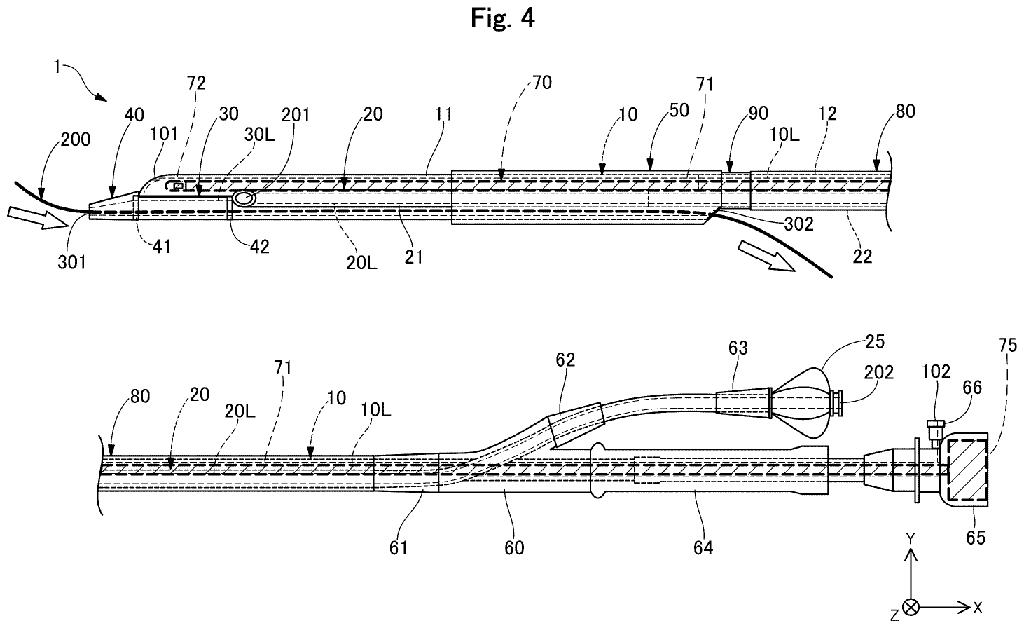

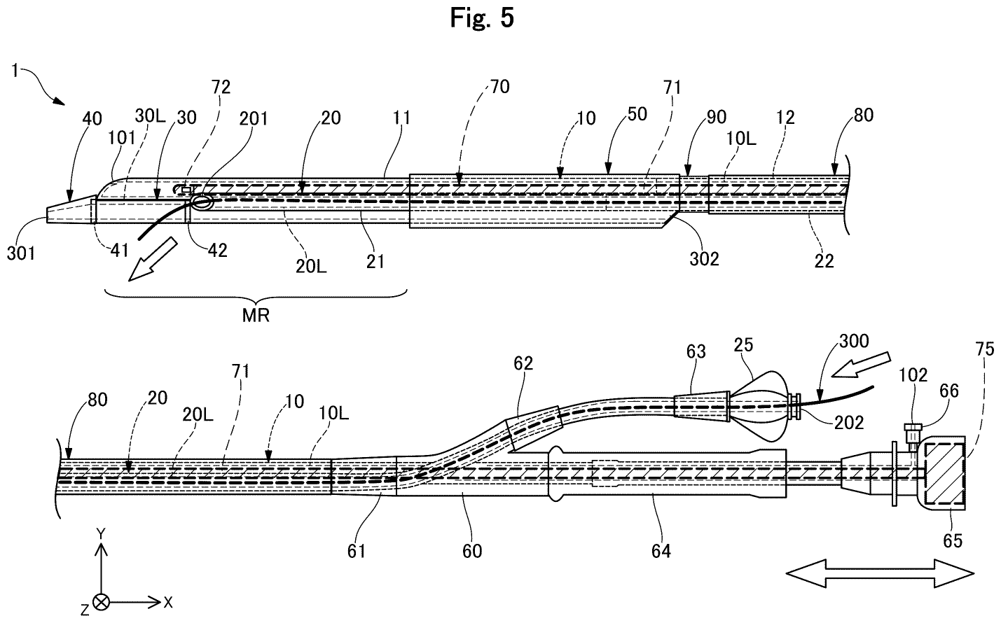

FIGS. 4 and 5 are diagrams illustrating a method of using the catheter 1. In the following steps a1 to a6, the case of recanalization of a CTO (lesion) generated in a blood vessel by the forward approach will be exemplified. The catheter 1 may be used in an inverse approach and may be used for operation other than recanalization of a CTO.

(a1) The professional inserts the work hose wire 200 into the blood vessel and delivers the distal end portion of the work hose wire 200 to the vicinity of the CTO. (a2) The professional inserts the proximal end portion of the work hose wire 200 from the distal end opening 301 of the catheter 1, passes it through the RX lumen 30L, and draws it out from the proximal end opening 302 of the catheter 1 (FIG. 4). (a3) The professional pushes the catheter 1 into the blood vessel along the work hose wire 200 and delivers the distal end portion of the catheter 1 to the vicinity of the CTO. In the step a3, the catheter 1 may be delivered to the vicinity of the CTO by passing the catheter 1 through a guiding catheter inserted into the blood vessel in advance along the work hose wire 200. (a4) While adjusting the position of the probe 72 of the sensor 70 within the movable range MR by gripping the connector 65 and sliding the connector 65 in the front-rear direction (FIG. 5: the direction of the white arrow), the professional checks the image displayed on the console terminal and thus matches the positions and orientations of the CTO and the distal end opening 201. The position means the position in the extending direction of the blood vessel, and the orientation means the orientation in the circumferential direction of the inner wall of the blood vessel. (a5) The professional inserts the distal end portion of a therapeutic device 300 from the proximal end opening 202 of the catheter 1, inserts the distal end portion into the OTW lumen 20L, and protrudes the distal end portion from the distal end opening 201 of the catheter 1 (FIG. 5). (a6) The professional treats the CTO using the therapeutic device 300 while adjusting the position of the probe 72 of the sensor 70 within the movable range MR as necessary and checking the image displayed on the console terminal. As described above, any device such as a plasma guide wire or a penetration guide wire can be used as the therapeutic device 300.

The sensor tube 10, the OTW tube 20, and the RX tube 30 are also collectively referred to as a “shaft” or a “catheter shaft”. The sensor tube 10 (the distal end side tube 11 and the proximal end side tube 12) corresponds to a “first tube”. The OTW tube 20 (the distal end side tube 21 and the proximal end side tube 22) corresponds to a “second tube”. The RX tube 30 corresponds to a “third tube”. The first outer tube 50 corresponds to a “fourth tube”. The second outer tube 80 corresponds to a “fifth tube”. The first outer tube 50 and the second outer tube 80 may individually or collectively be referred to as an “outer jacket”. The heat-shrinkable tube 90 corresponds to a “heat-shrinkable tube”. The distal end side tube 11 corresponds to a “first distal end side tube”, and the proximal end side tube 12 corresponds to a “first proximal end side tube”. The distal end side tube 21 corresponds to a “second distal end side tube”, and the proximal end side tube 22 corresponds to a “second proximal end side tube”. The sensor lumen 10L corresponds to a “lumen”. According to the present embodiment, “the same” and “equal” are not limited to a case of an exact match, and have a meaning of allowing a difference due to a manufacturing error or the like. In addition, “constant” is synonymous with “substantially constant”, and means substantially constant while allowing a deviation due to a manufacturing error or the like.

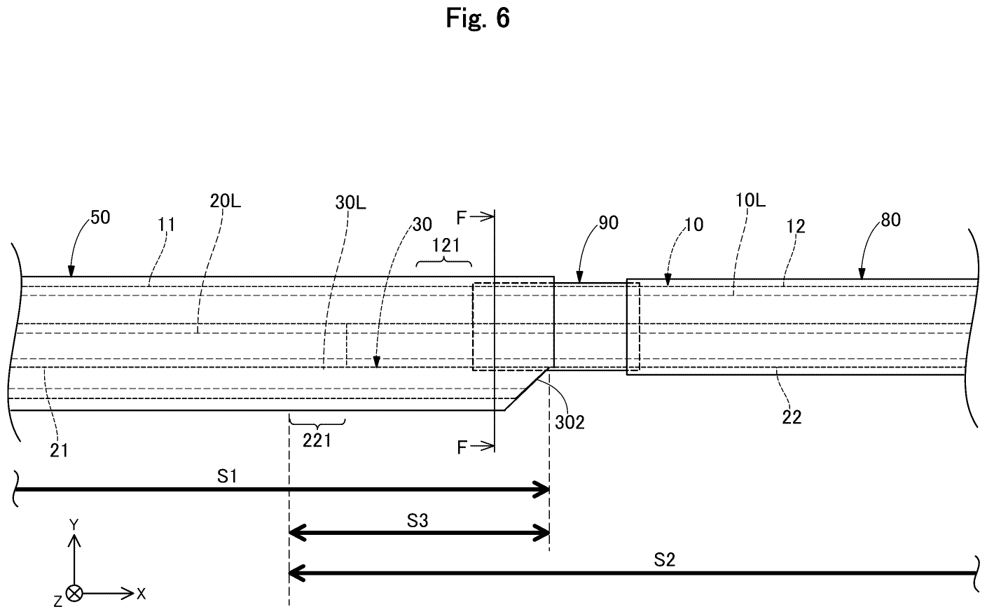

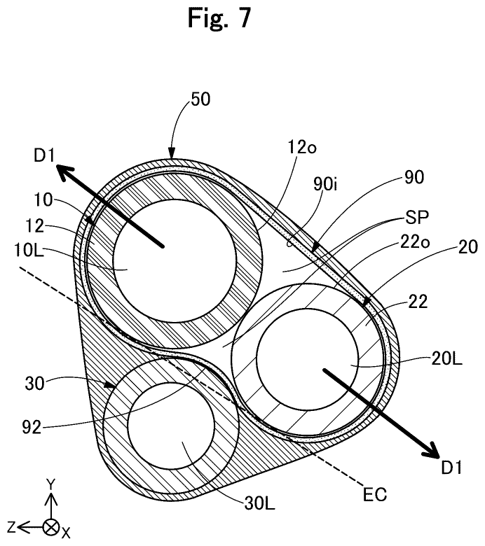

FIG. 6 is an enlarged view of a part of the catheter 1 in the vicinity of the heat-shrinkable tube 90. FIG. 7 is a transverse sectional view of the catheter 1 taken along the line F-F in FIG. 6. The relationship among the three tubes 10, 20, and 30 and the three tubes 90, 50, and 80 will be further described with reference to FIGS. 6 and 7.

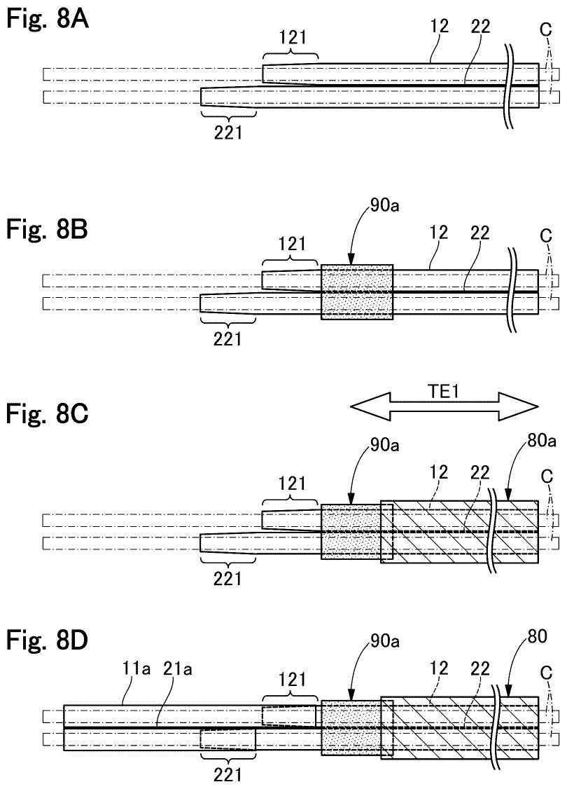

In the sensor tube 10 (first tube), a tapered portion 121, in which the outer diameter of the proximal end side tube 12 is reduced from the proximal end side toward the distal end side, is formed at the distal end portion of the proximal end side tube 12. The tapered portion 121 is covered with the proximal end portion of the distal end side tube 11. In other words, the distal end side tube 11 and the proximal end side tube 12 are provided to overlap each other in the range where the tapered portion 121 is provided. In the example of FIG. 6, the proximal end position of the distal end side tube 11 is the same as the distal end position of the heat-shrinkable tube 90. Similarly, in the OTW tube 20 (second tube), a tapered portion 221, in which the outer diameter of the proximal end side tube 22 is reduced from the proximal end side toward the distal end side, is formed at the distal end portion of the proximal end side tube 22. However, the tapered shape of the tapered portion 221 is not illustrated in FIG. 6. The tapered shape of the tapered portion 221 is illustrated in FIGS. 8A to 8D. The tapered portion 221 is covered with the proximal end portion of the distal end side tube 21. In other words, the distal end side tube 21 and the proximal end side tube 22 are provided to overlap each other in the range where the tapered portion 221 is provided. In FIG. 6, the proximal end position of the distal end side tube 21 is indicated by a broken line extending in the Y-axis direction on the OTW tube 20.

As illustrated in FIG. 6, the distal end position of the proximal end side tube 12 of the sensor tube 10 (the distal end position of the tapered portion 121) is different from the distal end position of the proximal end side tube 22 of the OTW tube 20 (the distal end position of the tapered portion 221). The distal end position of the proximal end side tube 22 of the OTW tube 20 is closer to the distal end side than the distal end position of the proximal end side tube 12 of the sensor tube 10.

Here, sections S1, S2, and S3 along the longitudinal direction of the catheter 1 are defined. The section S1 is a section from the first marker 41 to the proximal end of the RX tube 30. The section S2 is a section from the distal end of the proximal end side tube 22 of the OTW tube 20 (the distal end of the tapered portion 221) to the distal end of the first reinforcing member 61. The section S3 is a section from the distal end of the proximal end side tube 22 of the OTW tube 20 (the distal end of the tapered portion 221) to the proximal end of the RX tube 30. The section S1 is a section in which the distal end side tube 11, the distal end side tube 21, and the RX tube 30 are present, which are formed of a flexible material such as polyethylene resins described above. The section S1 is an area in which the rigidity of the catheter 1 is relatively low. The section S1 is an area where the melting points of the constituent members of the catheter 1 are relatively low. The section S2 is a section in which the proximal end side tube 12 and the proximal end side tube 22 are present, which are formed of a material having a high rigidity such as PEEK resins described above. The section S2 is an area where the rigidity of the catheter 1 is relatively high. The section S2 is an area where the melting points of the constituent members of the catheter 1 are relatively high.

The section S3 is an overlapping section of the section S1 and the section S2. In other words, the section S3 is a section in which the distal end side tube 11 and the RX tube 30, which have a low rigidity and a low melting point, and the proximal end side tube 12 and the proximal end side tube 22, which have a high rigidity and a high melting point, are mixed. For this reason, when the processing suitable for any one of the section S1 and the section S2 is performed in the section S1, there is a possibility that unintended variation occurs in the outer diameter or the physical properties of the catheter 1 and an appearance defect occurs. For example, when the processing suitable for the section S1 is performed in the section S3, a joining failure occurs in the proximal end side tube 12 and the proximal end side tube 22 having a high rigidity and a high melting point. Further, when the processing suitable for the section S2 is performed in the section S3, the distal end side tube 11 and the RX tube 30 having a low rigidity and a low melting point are deformed due to heat. In this regard, the catheter 1 according to the present embodiment solves these problems by using the heat-shrinkable tube 90.

As illustrated in FIG. 6, the heat-shrinkable tube 90 bundles a part of the distal end side of the proximal end side tube 12 of the sensor tube 10 (first tube) and a part of the distal end side of the proximal end side tube 22 of the OTW tube 20 (second tube). As illustrated in FIG. 7 (F-F transverse section), the sensor tube 10 and the OTW tube 20 are covered with the heat-shrinkable tube 90 in a state where parts of outer peripheral surfaces 12o and 22o are in contact with each other. Spaces SP are formed in areas surrounded by the outer peripheral surfaces 12o and 22o of the sensor tube 10 and the OTW tube 20 and an inner peripheral surface 90i of the heat-shrinkable tube 90. The spaces SP are present on both sides of the contact portion between the tubes 10 and 20. As illustrated in FIG. 7, the heat-shrinkable tube 90 is provided with a valley portion 92 that is recessed toward one of the spaces SP (specifically, the space SP closer to the RX tube 30).

As illustrated in FIG. 7, the RX tube 30 (third tube) is provided in contact with the valley portion 92 of the outer peripheral surface of the heat-shrinkable tube 90. The valley portion 92 of the heat-shrinkable tube 90 is formed during manufacturing when the RX tube 30 is pressed from the outer peripheral surface of the heat-shrinkable tube 90 toward the space SP. In FIG. 7, a common external tangent line EC on the RX tube 30 side is indicated by a broken line among the common external tangent lines of the outer peripheral surface 12o of the sensor tube 10 and the outer peripheral surface 22o of the OTW tube 20. In the transverse section illustrated in FIG. 7, the RX tube 30 intersects with the common external tangent line EC.

As illustrated in FIG. 7, the first outer tube 50 covers the heat-shrinkable tube 90 and the RX tube 30 provided in contact with the outer peripheral surface of the heat-shrinkable tube 90 at the portion where the heat-shrinkable tube 90 and the first outer tube 50 overlap with each other. The outer peripheral surface of the heat-shrinkable tube 90 and the outer peripheral surface of the RX tube 30 are integrally fixed to each other with the first outer tube 50 formed by melting.

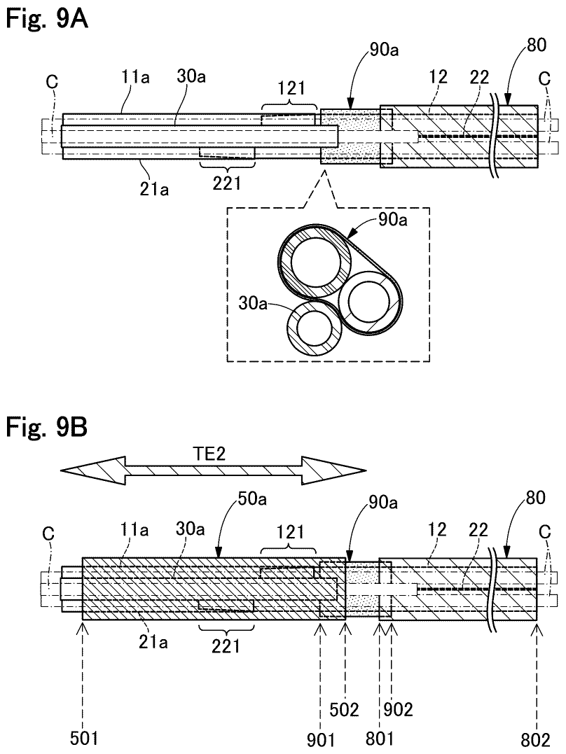

FIGS. 8A to 8D, 9A, and 9B are diagrams illustrating a method for manufacturing the catheter 1. In FIGS. 8A to 8D, 9A, and 9B, the first outer tube 50 is hatched with thin oblique lines, the second outer tube 80 is hatched with thick oblique lines, and the heat-shrinkable tube 90 is hatched with dots. In FIGS. 8A to 8D, 9A, and 9B, “a” is added to the end of the reference numeral of a member before a change whose shape is changed by heating or the like in the manufacturing process.

FIG. 8A illustrates a proximal end side tube placement step. As illustrated in FIG. 8A, the operator places the proximal end side tube 12 having the tapered portion 121 formed at the distal end portion thereof and the proximal end side tube 22 having the tapered portion 221 formed at the distal end portion thereof such that the distal end positions are shifted from each other. A cored bar C is inserted into each of the tubes 12 and 22.

FIG. 8B illustrates a heat-shrinkable tube placement step. As illustrated in FIG. 8B, the operator bundles the proximal end side tube 12 (first tube) and the proximal end side tube 22 (second tube) using a heat-shrinkable tube 90a. Specifically, the operator covers the proximal end side tube 12 and the proximal end side tube 22 with the heat-shrinkable tube 90a. At this time, the operator positions the distal end of the heat-shrinkable tube 90a at the proximal end of the tapered portion 121 of the proximal end side tube 12. As described above, by performing the heat-shrinkable tube placement step prior to a fifth tube placement step and a fourth tube placement step, the proximal end side tube 12 and the proximal end side tube 22 can be temporarily fixed to each other by the heat-shrinkable tube 90a, and the tubes can be prevented from being displaced.

FIG. 8C illustrates the fifth tube placement step. As illustrated in FIG. 8C, the operator covers the proximal end portion of the heat-shrinkable tube 90a and the proximal end side tube 12 (first tube) and the proximal end side tube 22 (second tube) exposed from the proximal end of the heat-shrinkable tube 90a with a second outer tube 80a (fifth tube). Then, the operator heats the range of the white arrow, in which the heat-shrinkable tube 90a and the second outer tube 80a are placed, at a first temperature TE1. The first temperature TE1 is a temperature at which the proximal end side tube 12 and the proximal end side tube 22 are not deformed and the second outer tube 80a is melted. By heating, the proximal end portion of the heat-shrinkable tube 90a shrinks. In addition, by heating, the second outer tube 80a is welded to the heat-shrinkable tube 90a and the tubes 12 and 22 to form a thick portion (FIG. 3E) formed by melting. The operator covers the second outer tube 80a with a heat-shrinkable tube for formation before heating and removes the heat-shrinkable tube for formation after heating (after melting).

FIG. 8D illustrates a distal end side tube placement step. As illustrated in FIG. 8D, the operator inserts a distal end side tube 11a to the cored bar C and slides the proximal end of the distal end side tube 11a to the same position as the proximal end of the tapered portion 121. Similarly, the operator inserts a distal end side tube 21a to the cored bar C and slides the proximal end of the distal end side tube 21a to the same position as the proximal end of the tapered portion 221.

FIG. 9A illustrates a step of placing an RX tube 30a in the fourth tube placement step. As illustrated in FIG. 9A, the operator prepares the RX tube 30a into which the cored bar C is inserted. Then, the operator places the RX tube 30a in a state where the RX tube 30a (third tube) is in contact with the outer peripheral surface of the heat-shrinkable tube 90a as illustrated in the balloon while the proximal end of the RX tube 30a is positioned at the position overlapping with the heat-shrinkable tube 90a (in other words, closer to the proximal end side than the tapered portion 121).

FIG. 9B illustrates a step of placing a first outer tube 50a in the fourth tube placement step. As illustrated in FIG. 9B, the operator covers the distal end portion of the heat-shrinkable tube 90a, the distal end side tube 11a (first tube) and the distal end side tube 21a (second tube), which are exposed from the distal end of the heat-shrinkable tube 90a, and the RX tube 30a (third tube) with the first outer tube 50a (fourth tube). Then, the operator heats the range of the arrow hatched with oblique lines, in which the heat-shrinkable tube 90a and the first outer tube 50a are placed, at a second temperature TE2. The second temperature TE2 is lower than the first temperature TE1 in the fifth tube placement step. The second temperature TE2 is a temperature at which the distal end side tube 11a, the distal end side tube 21a, and the RX tube 30a are not deformed and the first outer tube 50a is melted. By heating, the distal end portion of the heat-shrinkable tube 90a shrinks. In addition, by heating, the first outer tube 50a is welded to the heat-shrinkable tube 90a and the tubes 11a, 21a, and 30 to form a thick portion (FIGS. 3C, 3D, and 7) formed by melting. The operator covers the first outer tube 50a with a heat-shrinkable tube for formation before heating and removes the heat-shrinkable tube for formation after heating (after melting).

Then, the operator obliquely cuts the proximal end of the RX tube 30 and the first outer tube 50 covering the corresponding portion to form the proximal end opening 302. As illustrated in FIG. 9B, a distal end position 501 of the first outer tube 50 (fourth tube) is closer to the distal end side than a distal end position 901 of the heat-shrinkable tube 90. A proximal end position 802 of the second outer tube 80 (fifth tube) is closer to the proximal end side than a proximal end position 902 of the heat-shrinkable tube 90. Further, a proximal end position 502 of the first outer tube 50 (fourth tube) is closer to the distal end side than a distal end position 801 of the second outer tube 80 (fifth tube).

As described above, a suitable structure can be provided in a case where the sensor tube 10 (specifically, the proximal end side tube 12) as the first tube and the OTW tube 20 (specifically, the proximal end side tube 22) as the second tube have properties suitable (e.g., a high melting point) for being bundled by the heat-shrinkable tube 90 and the RX tube 30 as the third tube has a property unsuitable (e.g., a low melting point) for being bundled by the heat-shrinkable tube 90. That is, with this configuration, a suitable structure can be provided in a case where the catheter 1 has a section in which the distal end side tube 11 and the RX tube 30 having a low rigidity and a low melting point and the proximal end side tube 12 and the proximal end side tube 22 having a high rigidity and a high melting point are mixed as in the section S3 described in FIG. 6.

Further, the space SP is provided in the area surrounded by the outer peripheral surface 12o of the sensor tube 10 (first tube), the outer peripheral surface 22o of the OTW tube 20 (second tube), and the inner peripheral surface 90i of the heat-shrinkable tube 90 (FIG. 7). When the catheter 1 is used, the space SP makes it possible to suppress restrictions on the movement in a direction D1 (FIG. 7: the direction indicated by the thick arrow) in which the sensor tube 10 and the OTW tube 20 are adjacent to each other. Specifically, when a bending force in the direction D1 is applied during the use of the catheter 1, the sensor tube 10 and the OTW tube 20 can slide on each other in the heat-shrinkable tube 90 while the outer peripheral surfaces 12o and 22o slide on each other. As a result, the flexibility in the bending direction of the catheter 1 (medical device) can be improved.

Furthermore, since the heat-shrinkable tube 90 includes the valley portion 92, the catheter 1 (medical device) is thinner (FIG. 7) than that in a case where the heat-shrinkable tube 90 does not include the valley portion 92. In addition, since the RX tube 30 (third tube) is in contact with the valley portion 92, the catheter 1 (medical device) becomes much thinner (FIG. 7). Furthermore, since the RX tube 30 (third tube) intersects with the common external tangent line EC of the outer peripheral surface 12o of the sensor tube 10 (first tube) and the outer peripheral surface 22o of the OTW tube 20 (second tube), the catheter 1 (medical device) becomes much thinner (FIG. 7).

Furthermore, since the first outer tube 50 (fourth tube) that covers the heat-shrinkable tube 90 and the RX tube 30 (third tube) is provided, the heat-shrinkable tube 90 and the RX tube 30 can be bundled by the first outer tube 50. In addition, since the sensor tube 10 (first tube) has the sensor lumen 10L (first lumen) into which the sensor 70, which acquires image information, is inserted, the catheter 1 (medical device) can realize a procedure using image information (ultrasonic image) acquired from the sensor 70 (FIGS. 4 and 5).

Furthermore, the step (heat-shrinkable tube placement step: FIG. 8B) of bundling the sensor tube 10 (i.e., the proximal end side tube 12) as the first tube and the OTW tube 20 (i.e., the proximal end side tube 22) as the second tube and the step (fourth tube placement step: FIGS. 9A and 9B) of bundling the RX tube 30a as the third tube can be performed individually. In other words, according to the first embodiment, even when it is difficult to bundle the first, second, and third tubes 10, 20, and 30 at once due to factors such as the properties of the first, second, and third tubes 10, 20, and 30, the first, second, and third tubes 10, 20, and 30 can be integrated. Further, the strength of the catheter 1 (medical device) can be improved as compared with the case where the first, second, and third tubes 10, 20, and 30 are bundled at once.

Further, the first and second tubes 10 and 20 (specifically, the proximal end side tube 12 and the proximal end side tube 22) exposed from the proximal end of the heat-shrinkable tube 90a can be covered with the second outer tube 80a as the fifth tube together with the heat-shrinkable tube 90a (FIG. 8C). As a result, the strength of the portion of the catheter 1 (medical device) covered with the second outer tube 80 can be improved.

Further, the first and second tubes 10 and 20 (specifically, the distal end side tube 11a and the distal end side tube 21a) exposed from the distal end of the heat-shrinkable tube 90a can be covered with the first outer tube 50a as the fourth tube together with the RX tube 30a (third tube) and the heat-shrinkable tube 90a (FIGS. 9A and 9B). As a result, the strength of the portion of the catheter 1 (medical device) covered with the first outer tube 50 can be improved.

Further, the welding temperature (the second temperature TE2) in the step (fourth tube placement step) of covering with the fourth tube is lower than the welding temperature (the first temperature TE1) in the step (fifth tube placement step) of covering with the fifth tube, and therefore, when the RX tube 30a (third tube) having a melting point lower than the melting point of the sensor tube 10 (i.e., the proximal end side tube 12) as the first tube is used in the step of covering with the fourth tube, thermal deformation of the RX tube 30a can be suppressed.

### Second Embodiment

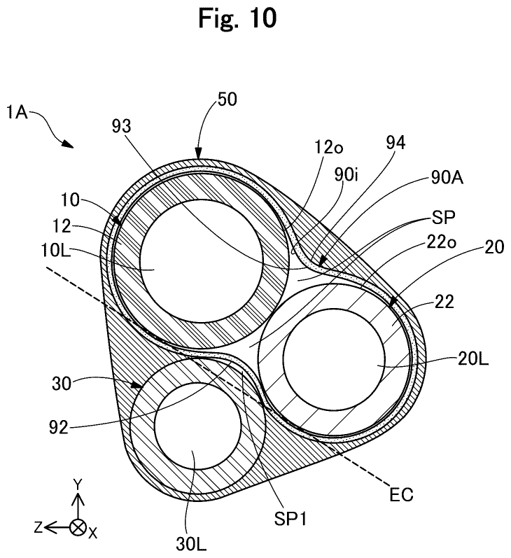

FIG. 10 is a transverse sectional view of a catheter 1A according to a second embodiment. FIG. 10 illustrates a transverse section of the catheter 1A taken along the line F-F of FIG. 6. The catheter 1A according to the second embodiment includes a heat-shrinkable tube 90A instead of the heat-shrinkable tube 90 in the configuration described in the first embodiment.

As illustrated in FIG. 10 (the F-F transverse section), the heat-shrinkable tube 90A includes a protruding portion 93 and a recess portion 94 in addition to the valley portion 92 described in the first embodiment. The protruding portion 93 is a portion of the heat-shrinkable tube 90A that protrudes toward the other space SP (i.e., the space SP farther from the RX tube 30). The recess portion 94 is a portion where the heat-shrinkable tube 90A is recessed toward the contact portion of the tubes 10 and 20 on the opposite side (outer peripheral surface side) of the protruding portion 93. As illustrated in FIG. 10, according to the second embodiment, the RX tube 30 (the third tube) is in contact with the outer peripheral surface of the heat-shrinkable tube 90A, but is not in contact with the valley portion 92. That is, a space SP1 is provided between the outer peripheral surface of the RX tube 30 and the valley portion 92.

As described above, the shape of the heat-shrinkable tube 90A can be variously changed. In addition, inside the heat-shrinkable tube 90A, the sensor tube 10 and the OTW tube 20 do not need to be in contact with each other, and the outer peripheral surfaces 12o and 22o may be separated from each other. In addition, the inner peripheral surface 90i of the heat-shrinkable tube 90A and the outer peripheral surface 12o of the sensor tube 10 do not need to be in contact with each other and may be separated from each other. Further, the inner peripheral surface 90i of the heat-shrinkable tube 90A and the outer peripheral surface 22o of the OTW tube 20 do not need to be in contact with each other and may be separated from each other. Further, the space SP does not need to be formed in the area between the inner peripheral surface 90i of the heat-shrinkable tube 90A and the outer peripheral surfaces 12o and 22o of the sensor tube 10 and the OTW tube 20. Also, in the catheter 1A according to the second embodiment as described above, the same effects as those of the first embodiment described above can be achieved.

### Third Embodiment

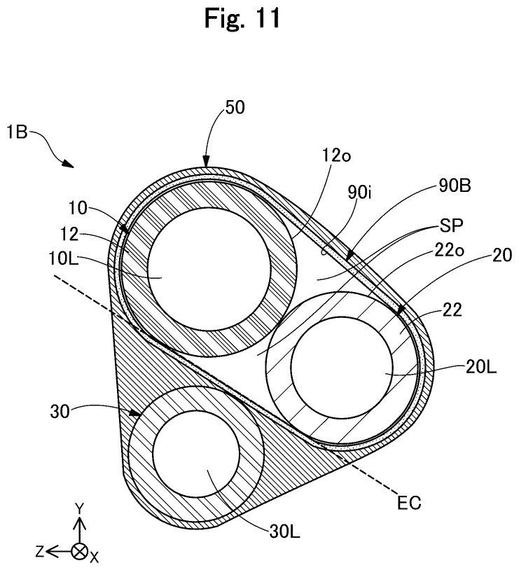

FIG. 11 is a transverse sectional view of a catheter 1B according to a third embodiment. FIG. 11 illustrates a transverse section of the catheter 1B taken along the line F-F of FIG. 6. The catheter 1B according to the third embodiment includes a heat-shrinkable tube 90B instead of the heat-shrinkable tube 90 in the configuration described in the first embodiment.

As illustrated in FIG. 11 (the F-F transverse section), the heat-shrinkable tube 90B does not include the valley portion 92 described in the first embodiment. As illustrated in FIG. 11, according to the third embodiment, the RX tube 30 (third tube) is in contact with the outer peripheral surface of the heat-shrinkable tube 90B, but is not in contact with the valley portion 92. Further, the RX tube 30 does not intersect with the common external tangent line EC of the outer peripheral surface 12o of the sensor tube 10 and the outer peripheral surface 22o of the OTW tube 20.

As described above, the shape of the heat-shrinkable tube 90B can be variously changed. In addition, the outer peripheral surfaces 12o and 22o may be separated from each other inside the heat-shrinkable tube 90B, and the inner peripheral surface 90i and the outer peripheral surface 12o (or the outer peripheral surface 220) do not need to be in contact with each other and may be separated from each other. The space SP does not need to be formed in the area between the inner peripheral surface 90i and each of the outer peripheral surfaces 12o and 22o. Also, in the catheter 1B according to the third embodiment as described above, the same effects as those of the first embodiment described above can be achieved.

### Fourth Embodiment

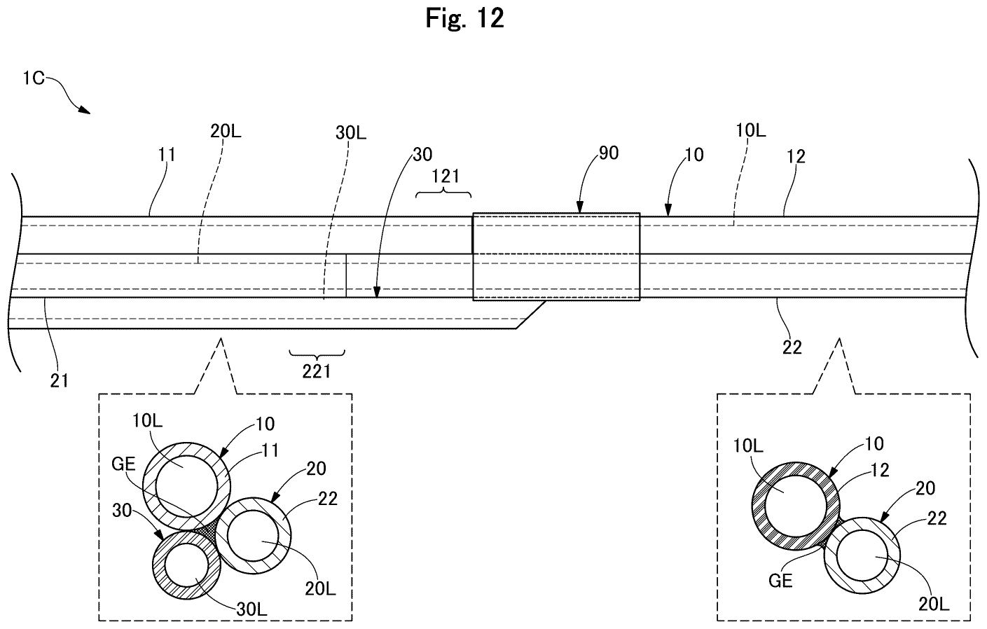

FIG. 12 is an enlarged view of a part of a catheter 1C according to a fourth embodiment in the vicinity of the heat-shrinkable tube 90. The catheter 1C according to the fourth embodiment does not include the first outer tube 50 and the second outer tube 80 in the configuration described in the first embodiment.

As illustrated in the left balloon in the lower part of FIG. 12, in the catheter 1C, the distal end side tube 11, the distal end side tube 21, and the heat-shrinkable tube 90 are bonded to each other with a bonding material GE. As illustrated in the right balloon in the lower part of FIG. 12, in the catheter 1C, the proximal end side tube 12 and the proximal end side tube 22 are bonded to each other with the bonding material GE. As the bonding material GE, any bonding material, such as a metal solder, for example, silver solder, gold solder, zinc, an Sn—Ag alloy, or an Au—Sn alloy, or an adhesive such as an epoxy-based adhesive can be used. Further, bonding by thermal welding may be adopted without using the bonding material GE.

As described above, the configuration of the catheter 1C can be variously changed, and the catheter 1C may be configured not to include at least one of the first outer tube 50 (fourth tube) and the second outer tube 80 (fifth tube). The catheter 1C according to the fourth embodiment described above can also achieve the same effects as those of the first embodiment described above.

### Modification of Present Embodiment

The disclosed embodiments are not limited to the above-described embodiments, and can be implemented in various modes without departing from the gist of the disclosed embodiments, and for example, the following modifications are also possible.

### [Modification 1]

According to the first to fourth embodiments described above, an example of the configurations of the catheters 1 and 1A to 1C has been described. Various modifications can be made to the configurations of the catheters 1 and 1A to 1C.

For example, the outer peripheral surfaces of the first outer tube 50, the heat-shrinkable tube 90, and the second outer tube 80, or the outer peripheral surface of the catheter 1 including these may be coated with a hydrophilic resin or a hydrophobic resin. For example, the sensor 70 is built in the sensor lumen 10L of the sensor tube 10 and is configured to be unremovable from the catheter 1. The sensor 70 may be configured to be removable from the catheter 1. That is, the catheter 1 does not need to include the sensor 70 as a constituent element.

For example, at least one of the distal tip 40, the first marker 41, and the second marker 42 may be omitted. For example, the shapes of the distal tip 40, the first marker 41, and the second marker 42 can be arbitrarily changed. The distal tip 40 may have a constant outer diameter from the distal end toward the proximal end, and the shape of the transverse section may be a non-circular symmetric shape. The first marker 41 and the second marker 42 may have a shape different from the annular shape (for example, a shape obtained by cutting an annular ring at an arbitrary angle, a linear shape, or a coil shape obtained by spirally winding a wire).

For example, the arrangement of the distal tip 40, the first marker 41, and the second marker 42 can be arbitrarily changed. The first marker 41 does not need to overlap with the distal tip 40 and may be provided at a position adjacent to the proximal end of the distal tip 40 or a position away from the proximal end of the distal tip 40. The second marker 42 may be provided at a position different from the position adjacent to the distal end of the distal end opening 201 of the OTW tube 20 (for example, a position away from the distal end opening 201). The first marker 41 and the second marker 42 may be provided on a tube (the sensor tube 10 or the OTW tube 20) different from the RX tube 30. The first marker 41 and the second marker 42 may be arranged on the same tube as described above, or may be arranged on different tubes.

For example, at least one of the first outer tube 50 and the second outer tube 80 may be omitted. For example, in the example of the embodiment described above, each of the first outer tube 50 and the second outer tube 80 is formed of one layer, but at least one of the first outer tube 50 and the second outer tube 80 may be formed of two or more layers. For example, in the example of the embodiment described above, the transverse sectional shape of the portion of the catheter 1 covered with the first outer tube 50 is a triangular shape with round corners, but may be any shape such as a circular shape or an elliptical shape. For example, in the example of the embodiment described above, the transverse sectional shape of the portion of the catheter 1 covered with the second outer tube 80 is an elliptical shape, but may be any shape such as a circular shape or a triangular shape with round corners. For example, at least one of the first outer tube 50 and the second outer tube 80 may have an outer shape along the contour of the outer peripheral surface of the tubes 10 and 20 (or the tubes 10, 20, and 30).

For example, the shapes of the branch connector 60, the first reinforcing member 61 to the third reinforcing member 63, the cylindrical member 64, the connector 65, and the connector 25 described above are merely examples, and may be arbitrarily changed. For example, at least a part of the branch connector 60, the first reinforcing member 61, the second reinforcing member 62, and the cylindrical member 64 may be configured as a single member or may be omitted. For example, the third reinforcing member 63 and the connector 25 may be configured as one member. For example, the cylindrical member 64 may include a mechanism (e.g., a scale or a stopper provided for each predetermined length in the longitudinal direction, or a scale or a stopper provided for each predetermined angle in the circumferential direction) that assists adjustment of at least one of the front-back position of the sensor 70 and the orientation of the sensor 70 in the circumferential direction.

For example, the methods for manufacturing the catheters 1 and 1A to 1C described in FIGS. 8A to 8D, 9A, and 9B are merely examples, and various modifications can be made. For example, the fourth tube placement step may be performed after the heat-shrinkable tube placement step, and then the fifth tube placement step may be performed. For example, pretreatment for processing or a separate step for placing other members which are not described may be further provided between the above-described steps. Further, for example, in the case of manufacturing the catheter 1C according to the fourth embodiment, the fourth tube placement step and the fifth tube placement step can be omitted.

### [Modification 2]

The configurations of the catheters 1 and 1A to 1C according to the first to fourth embodiments and the configurations of the catheters 1 and 1A to 1C according to the modification 1 may be appropriately combined. For example, in the configuration according to the fourth embodiment, the heat-shrinkable tubes 90A and 90B having the shape described in the second embodiment or the third embodiment may be combined.

### Aspects

(1) According to an aspect of the disclosed embodiments, a medical device is provided. The medical device includes a first tube, a second tube, a heat-shrinkable tube that bundles the first tube and the second tube, and a third tube that is in contact with an outer peripheral surface of the heat-shrinkable tube.

With this configuration, it is possible to provide a suitable structure in a case where the first tube and the second tube have properties suitable for being bundled by the heat-shrinkable tube and the third tube has a property unsuitable for being bundled by the heat-shrinkable tube.
(2) In the medical device according to the above-described aspect, a space may be provided in an area surrounded by an outer peripheral surface of the first tube, an outer peripheral surface of the second tube, and an inner peripheral surface of the heat-shrinkable tube. With this configuration, the space formed among the outer peripheral surface of the first tube, the outer peripheral surface of the second tube, and the inner peripheral surface of the heat-shrinkable tube makes it possible to suppress restrictions on the movement in the direction in which the first tube and the second tube are adjacent to each other. As a result, the flexibility in the bending direction of the medical device can be improved.
(3) In the medical device according to the above-described aspect, the heat-shrinkable tube may include a valley portion that is recessed toward the space. With this configuration, it is possible to make the medical device thinner compared to a case where the heat-shrinkable tube does not include a valley portion.
(4) In the medical device according to the above-described aspect, the third tube may be in contact with the valley portion. With this configuration, the medical device can be much thinner.
(5) In the medical device according to the above-described aspect, the third tube may intersect with a common external tangent line of an outer peripheral surface of the first tube and an outer peripheral surface of the second tube. With this configuration, the outer diameter of the medical device can be further reduced.
(6) In the medical device according to the above-described aspect, a melting point of the first tube may be higher than a melting point of the third tube.
(7) The medical device according to the above-described aspect may further include a fourth tube that covers the heat-shrinkable tube and the third tube. With this configuration, the heat-shrinkable tube and the third tube can be bundled by the fourth tube.
(8) In the medical device according to the above-described aspect, the first tube may have a lumen into which a sensor that acquires image information is inserted. With this configuration, a medical procedure using image information (ultrasonic image) acquired from the sensor can be realized by the medical device.
(9) In the medical device according to the above-described aspect, the first tube may include a first distal end side tube and a first proximal end side tube provided closer to a proximal end side than the first distal end side tube, the second tube may include a second distal end side tube and a second proximal end side tube provided closer to the proximal end side than the second distal end side tube, and the heat-shrinkable tube may bundle the first proximal end side tube and the second proximal end side tube. With this configuration, by using a tube (for example, a PEEK tube) having a higher rigidity and a higher melting point than the third tube as the first proximal end side tube and the second proximal end side tube and using a tube having a higher flexibility and a lower melting point than the first and second proximal end side tubes as the first distal end side tube and the second distal end side tube, it is possible to improve the torquability of the medical device while maintaining the flexibility of the distal end side of the medical device.
(10) According to an aspect of the disclosed embodiments, a method for manufacturing a medical device is provided. The method for manufacturing the medical device includes bundling a first tube and a second tube by using a heat-shrinkable tube, bringing a third tube into contact with an outer peripheral surface of the heat-shrinkable tube, and covering the heat-shrinkable tube and the third tube with a fourth tube. According to this manufacturing method, the step (heat-shrinkable tube placement step) of bundling the first tube and the second tube and the step (fourth tube placement step) of bundling the third tube can be performed individually. In other words, according to this manufacturing method, even when it is difficult to bundle the first, second, and third tubes at once due to factors such as the properties of the first, second, and third tubes, the first, second, and third tubes can be integrated. Further, the strength of the medical device can be improved as compared with the case where the first, second, and third tubes are bundled at once.
(11) The method for manufacturing the medical device according to the above-described aspect may further include, after bundling the first tube and the second tube by using the heat-shrinkable tube, covering the heat-shrinkable tube, the first tube exposed from a proximal end of the heat-shrinkable tube, and the second tube exposed from the proximal end of the heat-shrinkable tube, with a fifth tube. According to this manufacturing method, the first and second tubes exposed from the proximal end of the heat-shrinkable tube can be covered with the fifth tube together with the heat-shrinkable tube. As a result, the strength of the portion of the medical device covered with the fifth tube can be improved.
(12) In the method for manufacturing the medical device according to the above-described aspect, the covering with the fourth tube may include covering the heat-shrinkable tube, the first tube exposed from a distal end of the heat-shrinkable tube, the second tube exposed from the distal end of the heat-shrinkable tube, and the third tube with the fourth tube. According to this manufacturing method, the first and second tubes exposed from the distal end of the heat-shrinkable tube can be covered with the fourth tube together with the heat-shrinkable tube and the third tube. As a result, it is possible to improve the strength of the portion of the medical device covered with the fourth tube.
(13) In the method for manufacturing the medical device according to the above-described aspect, after covering with the fifth tube, the covering with the fourth tube may be performed, the covering with the fifth tube may include welding the fifth tube at a first temperature, a melting point of the third tube may be lower than a melting point of the first tube, and the covering with the fourth tube may include welding the fourth tube at a second temperature lower than the first temperature after a proximal end of the fourth tube is provided closer to a distal end side than a distal end of the fifth tube. According to this manufacturing method, the welding temperature (second temperature) in the step (fourth tube placement step) of covering with the fourth tube is lower than the welding temperature (first temperature) in the step (fifth tube placement step) of covering with the fifth tube, and therefore when the third tube having a melting point lower than the melting point of the first tube is used in the step of covering with the fourth tube, thermal deformation of the third tube can be suppressed.

Although the present mode has been described above based on the embodiments and the modifications, the embodiment of the above-described mode is intended to facilitate understanding of the present mode and does not limit the present mode. The present mode can be modified and improved without departing from the gist and the scope of the claims, and the present mode includes equivalents thereof. In addition, when the technical features are not described as essential in the present specification, the technical features can be appropriately deleted.

What is claimed is:

1. A medical device comprising:
a first tube;
a second tube;
a heat-shrinkable tube that bundles the first tube and the second tube; and
a third tube that is in contact with an outer peripheral surface of the heat-shrinkable tube.

2. The medical device according to claim 1, wherein
a space is provided in an area surrounded by an outer peripheral surface of the first tube, an outer peripheral surface of the second tube, and an inner peripheral surface of the heat-shrinkable tube.

3. The medical device according to claim 2, wherein
the heat-shrinkable tube includes a valley portion that is recessed toward the space.

4. The medical device according to claim 3, wherein
the third tube is in contact with the valley portion.

5. The medical device according to claim 1, wherein
in a transverse cross-section, the third tube intersects with a common external tangent line of an outer peripheral surface of the first tube and an outer peripheral surface of the second tube.

6. The medical device according to claim 1, wherein
a melting point of the first tube is higher than a melting point of the third tube.

7. The medical device according to claim 1, further comprising:
a fourth tube that covers the heat-shrinkable tube and the third tube.

8. The medical device according to claim 7, further comprising:
a fifth tube that covers a portion of the first tube and the second tube exposed from a proximal end of the heat-shrinkable tube.

9. The medical device according to claim 7, wherein
a transverse cross-section of a portion of the device covered by the fourth tube is substantially triangular.

10. The medical device according to claim 1, wherein
the first tube has a lumen into which a sensor that acquires image information is inserted.

11. The medical device according to claim 1, wherein
the first tube includes a first distal end side tube and a first proximal end side tube provided closer to a proximal end side than the first distal end side tube,
the second tube includes a second distal end side tube and a second proximal end side tube provided closer to the proximal end side than the second distal end side tube, and
the heat-shrinkable tube bundles the first proximal end side tube and the second proximal end side tube.

12. The medical device according to claim 1, wherein the first tube defines a sensor lumen, the second tube defines an over-the-wire lumen, and the third tube defines a rapid exchange lumen.

13. A method for manufacturing a medical device comprising:
bundling a first tube and a second tube by using a heat-shrinkable tube;
bringing a third tube into contact with an outer peripheral surface of the heat-shrinkable tube; and
covering the heat-shrinkable tube and the third tube with a fourth tube.

14. The method for manufacturing the medical device according to claim 13, further comprising:
after bundling the first tube and the second tube by using the heat-shrinkable tube, covering the heat-shrinkable tube, the first tube exposed from a proximal end of the heat-shrinkable tube, and the second tube exposed from the proximal end of the heat-shrinkable tube, with a fifth tube.

15. The method for manufacturing the medical device according to claim 13, wherein
the covering with the fourth tube includes covering a distal portion the heat-shrinkable tube, the first tube exposed from a distal end of the heat-shrinkable tube, the second tube exposed from the distal end of the heat-shrinkable tube, and the third tube.

16. The method for manufacturing the medical device according to claim 14, wherein
after covering with the fifth tube, the covering with the fourth tube is performed,
the covering with the fifth tube includes welding the fifth tube at a first temperature,
a melting point of the third tube is lower than a melting point of the first tube, and
the covering with the fourth tube includes welding the fourth tube at a second temperature lower than the first temperature after positioning a proximal end of the fourth tube distal to a distal end of the fifth tube.

17. The method for manufacturing the medical device according to claim 13, wherein the bundling creates a space between an inner surface of the heat-shrinkable tube and outer surfaces of the first and second tubes.

18. The method for manufacturing the medical device according to claim 13, wherein bringing the third tube into contact with the outer peripheral surface of the heat-shrinkable tube comprises pressing the third tube against the heat-shrinkable tube forms a valley portion therein.

19. A medical catheter shaft, comprising:
a first proximal tube defining a first lumen and having a first rigidity;
a second proximal tube defining a second lumen and having the first rigidity;
a heat-shrinkable tube securing the first proximal tube and the second proximal tube together in a side-by-side arrangement;
a third tube defining a third lumen and having a second rigidity lower than the first rigidity, wherein a portion of the third tube is positioned adjacent to an outer surface of the heat-shrinkable tube; and
an outer jacket covering at least a portion of the heat-shrinkable tube and the third tube.
